(19) United States
(12) Patent Application Publication
(10) Pub. No.: US 2006/0052791 A1
(43) Pub. Date: Mar. 9, 2006

(54) Surgical positioning and holding device

(75) Inventors: Thomas Hagen, Tuttlingen (DE); Jan Reich, Hochemmingen (DE)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/210,156

(22) Filed: Aug. 22, 2005

Related U.S. Application Data
(63) Continuation of application No. PCT/EP04/01347, filed on Feb. 13, 2004.

(30) Foreign Application Priority Data
Feb. 28, 2003 (DE) 103 09 987.5

(51) Int. Cl. A61F 5/00

(52) U.S. Cl. 606/86

(57) ABSTRACT

In order to improve a surgical positioning and holding device for positioning and holding a guide for a surgical machining tool, with at least one fastening element for fixation to a bone to be machined and with a platform held on the at least one fastening element so as to hold the guide, so that anchoring surfaces may be prepared in a simple manner and with a high degree of precision on a bone to be machined, it is proposed that the guide be mounted for rotation about a first axis of rotation and be designed such that at least one surface concentric with the first axis of rotation may be prepared with the machining tool guided in or held on the guide.

This application is a continuation of international application number PCT/EP2004/001347 filed on Feb. 13, 2004.

The present disclosure relates to the subject matter disclosed in international application number PCT/EP2004/001347 of Feb. 13, 2004 and German application number 103 09 987.5 of Feb. 28, 2003, which are incorporated herein by reference in their entirety and for all purposes.

### BACKGROUND OF THE INVENTION

The present invention relates to a surgical positioning and holding device for positioning and holding a guide for a surgical machining tool, with at least one fastening element for fixation to a bone to be machined and with a platform held on the at least one fastening element so as to hold the guide mounted for rotation about a first axis of rotation.

Such devices are used, for example, in operations on joints, in which parts of a damaged joint are replaced by artificial joint components. For this purpose, the device is fixed with the at least one fastening element on the bone to be machined, and an anchoring surface for the artificial joint component is prepared using a machining tool guided in the guide. Herein, in particular, flat anchoring surfaces are formed on bones by partial resection. An example of preparation of a spherical anchoring surface is described in U.S. Pat. No. 5,314,482. Herein it is disadvantageous that the machining tool has to be advanced towards the bone to be machined from the front because this makes complete opening of the damaged joint necessary.

The object underlying the present invention is, therefore, to so improve a surgical positioning and holding device of the kind described at the outset that anchoring surfaces may be prepared in a simple manner and with a high degree of precision on a bone to be machined.

### SUMMARY OF THE INVENTION

This object is accomplished in accordance with the invention in a surgical positioning and holding device of the kind described at the outset in that the guide is mounted for rotation about a first axis of rotation and is designed such that at least one surface concentric with the first axis of rotation may be prepared with the machining tool guided in or held on the guide.

Cylindrical surfaces may be prepared on a bone in an extremely simple way with such a device. Owing to the special orientation of the axis of rotation, it is, for example, possible to work on a condyle on a femur from a lateral or medial direction. Therefore, complete opening of the joint to be worked on is not necessary, but rather such surgery may also be performed by a minimally invasive technique or by a miniarthrotomy.

To ensure a particularly secure hold of the device on the bone to be worked on, it is advantageous for a second fastening element to be provided for fixation to the bone to be worked on and for the second fastening element to be guided and/or held on the platform.

To join the platform in a simple way to the at least one fastening element, the platform may comprise at least one fastening element receptacle for receiving the at least one fastening element. The fastening element may be inserted into the fastening element receptacle and optionally additionally secured therein against relative movement.

A particularly simple configuration of the device is obtained when the at least one fastening element receptacle comprises a bore. In particular, this may be a blind hole bore.

To join the platform in a simple way to several fastening elements, it is advantageous for longitudinal axes of at least two fastening element receptacles to be aligned parallel to each other. The platform may then be guided onto fastening elements fixed in the bone to be machined in the direction of the longitudinal axes into or onto the fastening element receptacles.

In accordance with a preferred embodiment of the invention, provision may be made for the longitudinal axes of the at least two fastening element receptacles to extend parallel or almost parallel to the first axis of rotation. If, for example, the at least one fastening element is anchored in a navigationally assisted manner in the bone to be machined, a direction of the first axis of rotation can then be precisely or roughly specified.

It is advantageous for a bearing shaft to be provided on the platform and for the bearing shaft to define the first axis of rotation. In this way, the first axis of rotation is optically immediately recognizable. The bearing shaft may be arranged movably relative to the platform or stationarily thereon.

To allow fine adjustment of the first axis of rotation relative to the at least one fastening element, the bearing shaft is mounted on the platform so as to be displaceable in a first direction of displacement relative to the at least one fastening element. The first direction of displacement may be optionally selected, in particular, parallel or transversely to the first axis of rotation.

For fine adjustment of the first axis of rotation relative to the at least one fastening element, the bearing shaft may be mounted on the platform so as to be displaceable in a second direction of displacement relative to the at least one fastening element. In particular, the first and second directions of displacement may be oriented at right angles to each other.

A position of the first axis of rotation relative to the at least one fastening element is adjustable for a third degree of freedom when the bearing shaft is mounted on the platform so as to be displaceable about a second axis of rotation relative to the at least one fastening element. An angle of inclination of the first axis of rotation relative to a longitudinal axis of the at least one fastening element is thereby adjustable.

The first and second axes of rotation are preferably oriented at right angles to each other. The first axis of rotation can thus be inclined relative to the at least one fastening element, so that the surface to be prepared on the bone to be machined can be at an incline relative to a longitudinal axis of the at least one fastening element.

A particularly simple design is obtained for the device when it comprises an articulated arm mounted for rotation about the first axis of rotation, when one end of the articulated arm is mounted on the bearing shaft so as to be rotatable about the first axis of rotation and when another end of the articulated arm carries the guide. A compass-type construction of the device may be realized in a particularly simple way with such an articulated arm.

A particularly good hold and a particularly good guidance of the machining tool on the device are obtained by the guide comprising a sleeve for receiving the machining tool. For example, a machining tool in the form of a milling cutter or a drill may be guided almost without any play in the sleeve, so that cylindrical surfaces may be prepared with a high degree of precision on the bone.

It is advantageous for the sleeve to be rotatably mounted on the articulated arm. This results in a decrease in wear of the device. The sleeve is advantageously mounted by means of ball bearings on the articulated arm.

The first axis of rotation may be altered in its position relative to the at least one fastening element in a simple way when a first linear drive is provided on the platform in order to displace the bearing shaft in the first direction of displacement relative to the at least one fastening element.

A particularly simple design is obtained for the device when the first linear drive is a spindle drive with a first threaded spindle and a first drive knob and when a longitudinal axis of the first threaded spindle defines the first direction of displacement. Only a minimum number of components is required for a spindle drive, which simplifies the construction of the device.

It is advantageous for a second linear drive to be provided on the platform in order to displace the bearing shaft in the second direction of displacement relative to the at least one fastening element. A position of the first axis of rotation relative to the at least one fastening element may be adjusted in a simple way with the second linear drive.

To simplify a construction of the device it is advantageous for the second linear drive to be a second spindle drive with a second threaded spindle and a second drive knob and for a longitudinal axis of the second threaded spindle to define the second direction of displacement.

For the device to be of particularly compact design, the longitudinal axis of the second threaded spindle may define the second axis of rotation. For example, the threaded spindle could serve as bearing shaft for a pivotal movement about the second axis of rotation.

To realize a pivotal movement of the first axis of rotation relative to the at least one fastening element in a simple way, an eccentric drive may be provided on the platform in order to pivot the bearing shaft about the second axis of rotation relative to the at least one fastening element.

A particularly simple construction is obtained when the eccentric drive comprises a rotational member mounted eccentrically about a third axis of rotation and when the third axis of rotation extends parallel to the second axis of rotation.

In operations on knee joints, for example, the problem may arise that the first axis of rotation has to be positioned so as to intersect an area of attachment of collateral ligaments, muscles, tendons or these themselves. Therefore, if the first axis of rotation were defined by the at least one fastening element, this would result in damage to the collateral ligaments. It is, therefore, advantageous for the at least one fastening element to be spaced from the axis of rotation. In particular, the device may be so designed that the fastening elements are arranged in an area of the bone to be machined that is remote from the area of attachment of the collateral ligaments, so that no tendons, muscles or ligaments are damaged. With this construction, the first axis of rotation may nevertheless intersect ligaments or the like.

In accordance with a preferred embodiment of the invention it may be advantageous for the guide to be securable in a rotational position relative to the platform. Depending on its design, the guide may then itself define an axis of rotation for a machining tool, for example, a cylindrically curved saw blade, with which a likewise cylindrical surface may be prepared on a bone to be machined.

In particular, it is advantageous for the guide to define a fourth axis of rotation. In this way, surfaces concentric with the fourth axis of rotation may be prepared on a bone to be machined with corresponding machining tools, for example, with cylindrically curved saw blades.

A particularly compact design is obtained when a width of the platform in the second direction of displacement is 30 mm at most. The device is then also suitable for minimally invasive operations.

It is advantageous for a spacing of the guide from the first axis of rotation to lie in a range of from 15 mm to 50 mm. Radii of curvature of the surface to be machined can thus be realized in the given range or even smaller ones given a corresponding diameter of the machining tool. In addition, an overall height of the device is thus reduced.

To be able to use the device particularly universally, a set of articulated arms of different lengths may be provided, in accordance with a preferred embodiment of the invention, and each articulated arm may have a different spacing between the axis of rotation and the guide. Depending on the size of the bone to be machined, an articulated arm of optimum length may be selected and joined to the platform for guiding the machining tool.

It is advantageous for a reference element for navigation control to be provided on the device. Surfaces may thus be prepared in a navigationally assisted manner on the bone to be machined. In particular, when the at least one fastening element has already been anchored in a navigationally assisted manner on the bone to be machined, a fine adjustment of the first axis of rotation relative to the at least one fastening element may be carried out under navigational control.

It is advantageous for the guide to be displaceable in a direction parallel to the first axis of rotation relative to the platform. Such an arrangement enables a further possibility for adjusting the guide relative to the fastening elements. In particular, whenever adjustability of the bearing shaft relative to the securing pins is not possible or only possible with difficulty, the guide may be brought in this way into a desired position.

It is advantageous for the fourth axis of rotation defined by the guide to extend at right angles to the first axis of rotation. This arrangement makes it possible to work on a bone with a machining tool from the front, for example, with a face milling cutter. In this way, a surface concentric with the first axis of rotation may be made.

The fourth and first axes of rotation preferably intersect each other. A path concentric with the first axis of rotation may thereby be directly described with an end of a machining tool.

The following description of preferred embodiments of the present invention serves in conjunction with the drawings for further explanation.

### BRIEF DESCRIPTION OF THE DRAWINGS

FIG. 1 is a perspective view of an aligning instrument according to the invention secured on a bone to be worked on;

FIG. 2 is a lateral view of the aligning instrument secured on the bone to be worked on;

FIG. 3 is a sectional view of the aligning instrument;

FIG. 4 is a sectional view of the aligning instrument along line 4-4 in FIG. 3;

FIG. 5 is a sectional view of the aligning instrument along line 5-5 in FIG. 3;

FIG. 6 is a sectional view similar to FIG. 3 with the aligning instrument in a pivoted position;

FIG. 7 is a perspective view of a second embodiment of an aligning instrument;

FIG. 8 is a further perspective view of the second embodiment of an aligning instrument; and

FIG. 9 is a cross-sectional view of the second embodiment of an aligning instrument.

### DETAILED DESCRIPTION OF THE INVENTION

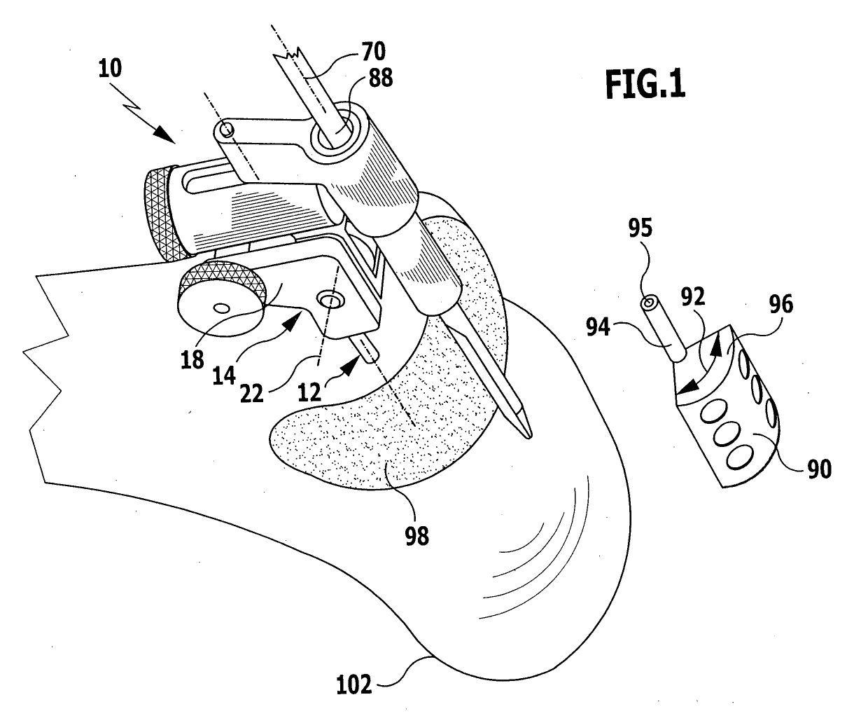

FIG. 1 shows a surgical positioning and holding device according to the invention, which comprises an aligning instrument generally designated by reference numeral 10 and two bone pins 12.

The aligning instrument 10 comprises two frame parts mounted for pivotal movement relative to each other, namely a holding frame 14 connected to the bone pins 12 and a bearing frame 16. The holding frame 14 comprises two flat, L-shaped side walls 18 which are arranged parallel to each other and are connected to each other by a connecting plate 20. The substantially L-shaped bearing frame 16 is mounted between the side walls 18 so as to be pivotable about a pivot axis 22 relative to the holding frame 14.

The pivot axis 22 is defined by a threaded spindle 24 of a spindle drive generally designated by reference numeral 25. The threaded spindle 24 is rotationally fixedly connected to the side walls 18 and is provided with an external thread in an area between the two side walls 18. It also extends through a bore 26 of a leg 28 of the bearing frame 16, which is held between the side walls 18. The leg 28 is provided transversely to the bore 26 with an opening 30 of rectangular parallelepiped shape, in which an adjusting wheel 32 provided with an internal thread 34 is arranged. The internal thread 34 corresponds to an external thread 36 of the threaded spindle 24. A width of the leg 28 in the direction of the pivot axis 22 is smaller than a distance between the side walls 18, so that a sideways movement of the bearing frame 16 relative to the holding frame 14 is made possible by means of the spindle drive 25, i.e., by turning the adjusting wheel 32 on the threaded spindle 24. The threaded spindle 24 thus forms together with the adjusting wheel 32 a linear drive in the form of the spindle drive 25.

A hollow-cylindrical bearing sleeve 38 forms a second leg of the bearing frame 16 extending at right angles to the leg 28. An axis of symmetry 40 of the bearing sleeve 38 is oriented perpendicular to the pivot axis 22. Parallel to the axis of symmetry 40 the bearing sleeve 38 is provided with a longitudinal slot 42 which extends over almost the entire length of the bearing sleeve 38 and through which a cylindrical bearing bolt 44 projects. It is rotationally fixedly connected to a cylindrical displacement member 46 which is guided in the bearing sleeve 38. An outer diameter of the displacement member 46 is only insignificantly smaller than an inner diameter of the bearing sleeve 38, so that the displacement member 46 can only be displaced in the direction of the axis of symmetry 40 in the bearing sleeve 38. A rotation of the displacement member 46 in the bearing sleeve 38 is prevented by the bearing bolt 44 extending through the longitudinal slot 42.

The displacement member 46 is also rotationally fixedly connected to a threaded bolt 48 which projects through a front bore 50 of a front face 52 of the bearing sleeve 38. Inserted in the front bore 50 with a positive fit is a threaded sleeve 54 which is provided with an internal thread and is rotationally fixedly connected to a knurled head 56 lying on the outside against the front face 52. The threaded sleeve 54 projects somewhat into the bearing sleeve 38 and is secured with a retaining ring 58 against axial displacement in the direction of the axis of symmetry 40. The axis of symmetry 40 coincides with an axis of symmetry of the threaded bolt 48. By turning the knurled head 56 the threaded bolt 48 is moved in the direction of the axis of symmetry 40, so that the displacement member 46 is displaced linearly in the bearing sleeve 38. In this way a linear drive 60 in the form of a spindle drive is formed.

A lever 62 of rectangular parallelepiped shape is provided at one end thereof with a bore 64 in which the bearing bolt 44 is inserted. The lever 62 is thus pivotable about the bearing bolt 44 forming a bearing shaft, namely about an axis of rotation 66 defined by the bearing bolt 44. At its other end the lever 62 is integrally connected to a guide sleeve 68 whose axis of symmetry defines an axis of rotation 70. The axis of rotation 70 extends parallel to the axis of rotation 66. Inserted into the guide sleeve 68 is a further bearing sleeve 72 whose axis of symmetry coincides with the axis of rotation 70. The bearing sleeve 72 is somewhat more than twice as long as the guide sleeve 68. It is rotationally fixedly connected to the guide sleeve 68. It is also conceivable for the bearing sleeve 72 to be mounted on the guide sleeve 68 by means of a ball bearing.

An eccentric bolt 74 is rotatably held in bores 76 in the side walls 18 parallel to the threaded spindle 24. Arranged between the side walls 18 is a supporting cylinder 78 which is rotationally fixedly connected to the eccentric bolt 74. An adjusting wheel 80 is arranged at one end of the eccentric bolt 74. Another end is provided with a bolt head 82. A movement in the direction of an eccentric axis 84 defined by a longitudinal axis of the eccentric bolt 74 is prevented by the eccentric bolt 74 being held at both sides at one side wall 18 by the bolt head 82 and the supporting cylinder 78 and at the other side wall 18 by the supporting cylinder 78 and the adjusting wheel 80. A circumferential wall of the supporting cylinder 78 forms a supporting surface 86 for the bearing sleeve 38. By turning the adjusting wheel 80 the supporting cylinder 78 is pivoted about the eccentric axis 84, so that a spacing of the bearing sleeve 38 resting against the supporting cylinder 78 from the eccentric axis 74 is altered and thereby brings about a pivoting movement of the bearing sleeve 38 and thus of the bearing frame 16 about the pivot axis 22. The axis of rotation 66 is also inclined relative to the pivot axis 22 by this pivoting.

A machining tool, for example, a milling cutter 88 or a saw blade 90 may be guided in the bearing sleeve 72. The saw blade 90 is curved in the shape of a section of a cylinder wall and extends over an angular range 92 of approximately 100°. Arranged concentrically with the saw blade 90 on a cover plate 96 is a holding pin 94. The holding pin 94 may likewise be guided in the bearing sleeve 72.

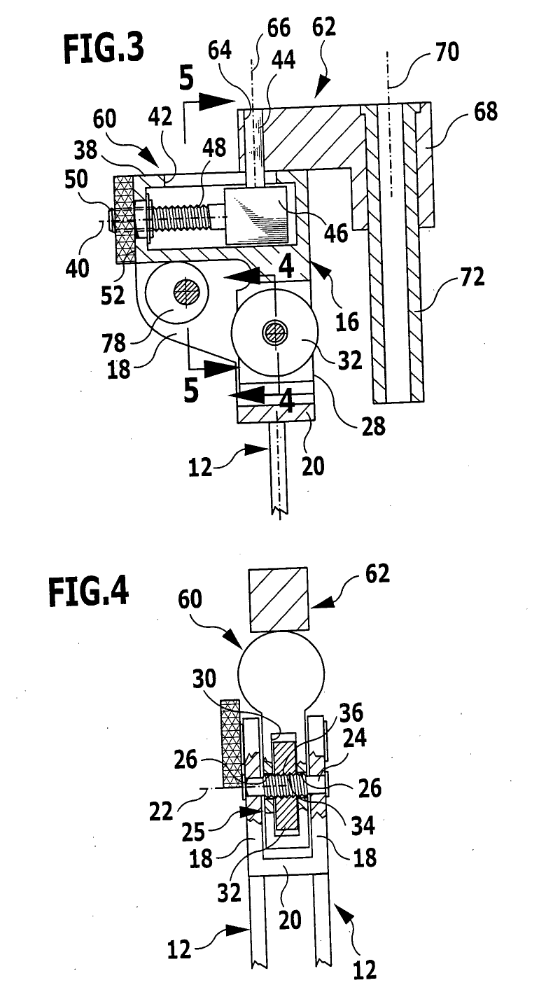

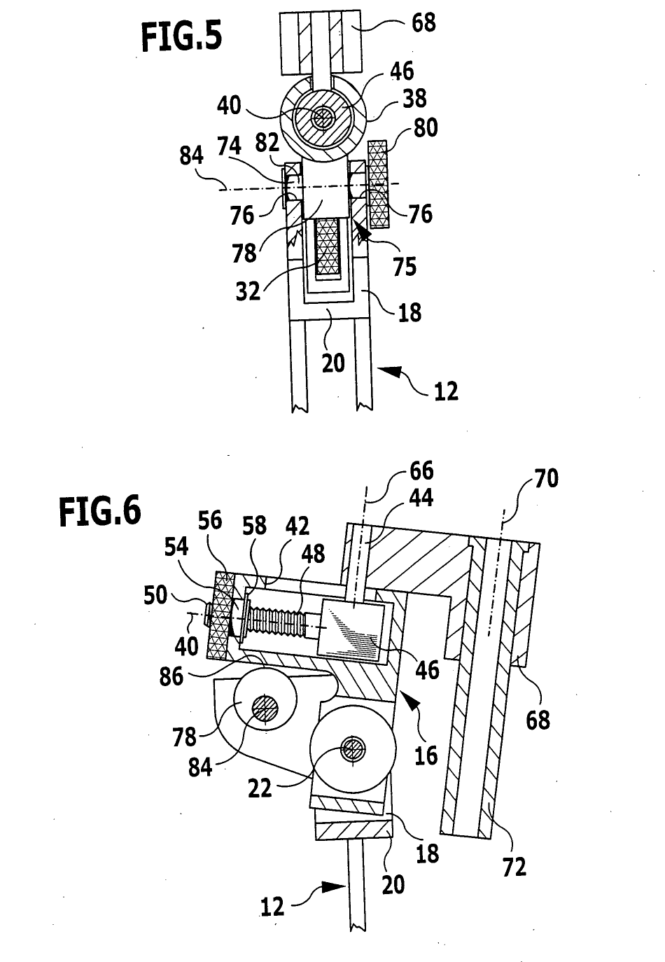

Use of the aligning instrument 10 is explained hereinbelow in conjunction with FIGS. 1 to 6, by way of example, in conjunction with preparation of a cylindrical anchoring surface 98 for anchoring an artificial condyle 100, which is to replace a partially damaged, natural condyle on a femur 102.

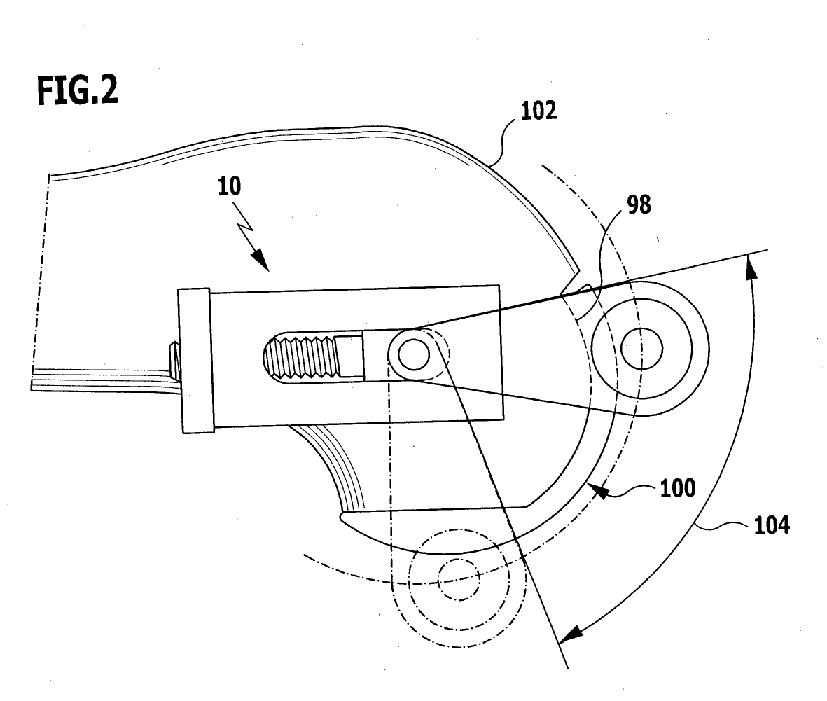

The aligning instrument 10 is advanced with the two bone pins 12 either laterally or medially, depending on the damaged condyle is to be replaced, towards the femur 102. This may be done in a navigationally assisted manner. The bone pins 12 are driven into the femur 102 and the aligning instrument is anchored in this way. A machining tool, for example, the milling cutter 88, is inserted into the bearing sleeve 72 and made to rotate so as to machine the femur 102 and simultaneously pivoted within an angular range 104 of approximately 80 to 90° by pivoting the lever 62 about the axis of rotation 66, as shown in FIG. 2. The femur 102 is thus resected in the desired manner.

If necessary, a position of the axis of rotation 66 relative to the femur 102 may be readjusted before preparing the anchoring surface 98. To do so, an adjustment in the direction of the axis of symmetry 40 may be made by means of the linear drive 60. Furthermore, a linear displacement of the bearing bolt 44 relative to the bone pins 12 may be carried out by turning the adjusting wheel 32 in the direction of the pivot axis 22.

In addition, the axis of rotation 66 may be altered in its inclination relative to the bone pins 12 by turning the adjusting wheel 80, which, as described hereinabove, brings about a pivoting of the bearing frame 16 about the pivot axis 22.

For use with the cylindrical saw blade 90 described hereinabove the lever 62 may be fixed in a pivoted position. In the case of differently shaped saw blades 90, a pin borehole 95 extending through the holding pin 94 in the longitudinal direction thereof may also be provided for receiving the bearing bolt 44. Instead of the lever 62, the holding pin 94 can thus be pivotably mounted on the bearing bolt 44.

To obtain anchoring surfaces with different radii, several different levers 62 are provided, with the spacing between the axis of rotation 66 and the axis of rotation 70 varying in each case. Depending on the condyle 100 to be implanted, a corresponding lever 62 will be selected for preparation of the anchoring surface 98.

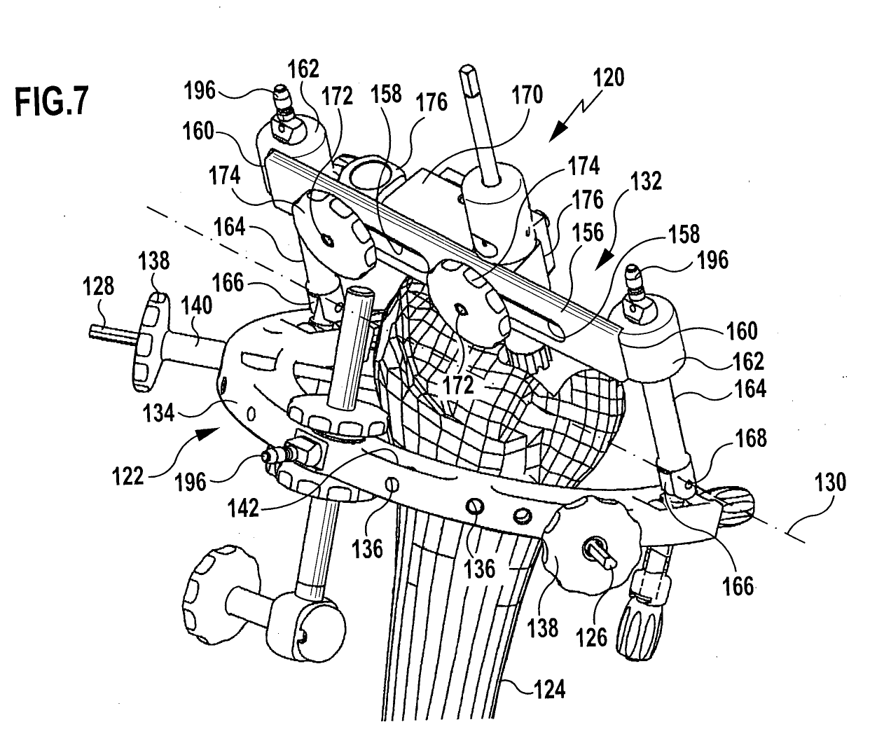

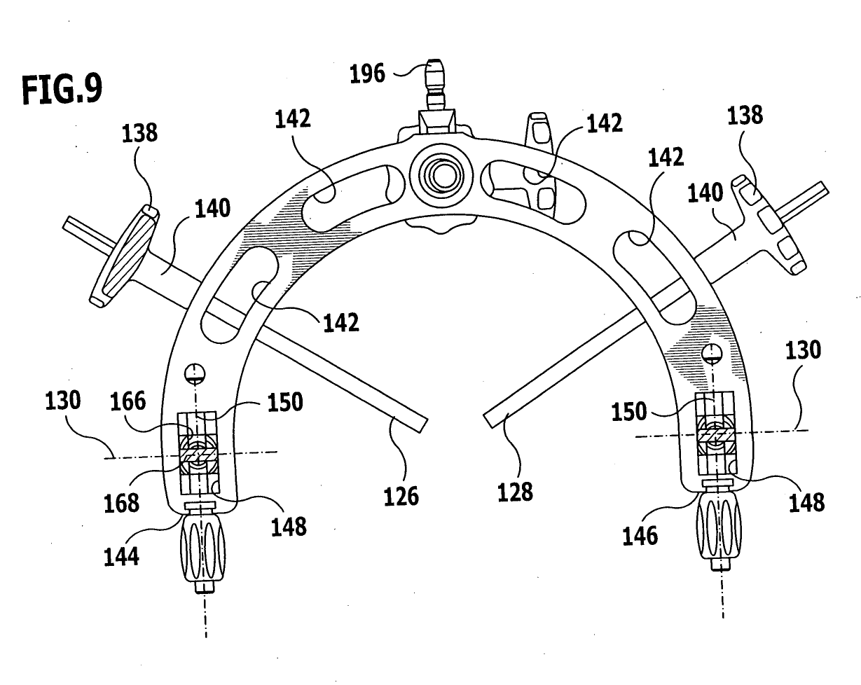

A second aligning instrument, generally designated by reference numeral 120, is shown in FIGS. 7 to 9. It comprises a platform, generally designated by reference numeral 122, which may be held on a femur 124 by means of two bone pins 126 and 128 which include approximately an angle of 120° between them. A pivot bracket, generally designated by reference numeral 132, is mounted on the platform so as to be pivotable about a pivot axis 130.

Both the platform 122 and the pivot bracket 132 are substantially symmetrical in relation to a plane of symmetry perpendicular to the pivot axis 130.

The platform 122 comprises a half ring-shaped frame 134 defining a frame plane. There are provided parallel to the frame plane a plurality of pin holes 136 through which the bone pins 126 and 128 are insertable, so that these are aligned in a plane at an angle of 120° relative to each other. Ends of the bone pins 126 and 128 projecting from the femur 124 are provided with a screw thread onto which a threaded sleeve 140 provided with a turning knob 138 is screwable and fixes the frame 134 on the two bone pins 126 and 128. Elongated openings 142 are provided on the frame 134 transversely to longitudinal axes of the bone pins 126 and 128. Free ends 144 and 146 of the frame 134 have an elongated opening 148 extending away from the ends 144 and 146. Inserted in each of these openings 148 is a shaft 150 extending away from the ends 144 and 146. The shaft 150 carries a bearing sleeve 152 displaceable on the shaft and defines a longitudinal axis 151.

A bearing bolt 154 is arranged on the bearing sleeve 152 and protrudes at right angles therefrom. The substantially U-shaped pivot bracket 132 is mounted on the bearing bolt 154 so as to be pivotable about the pivot axis 130. The pivot bracket 132 comprises an elongated plate 156 of rectangular parallelepiped shape, which is provided with two elongated holes 158 arranged symmetrically.

A cylinder 162 is arranged at free ends 160 of the plate 156 with its longitudinal axis transversely to the longitudinal direction of the plate 156. It continues into a cylindrical rod 164 of decreased diameter, which carries at its free end a bearing groove 166 which is mounted by means of a joint pin 168 on the bearing bolt 154 so as to be pivotable about the pivot axis 130.

A bearing slide 170 of rectangular parallelepiped shape is displaceable parallel to the elongated holes 158 and to the longitudinal direction of the plate 156. It carries two set screws 172 extending parallel through the elongate holes 158 with a knurled nut 174 screwed onto each one. The bearing slide 170 can be clamped to the plate 156 by means of the knurled nuts 174. In order to change a position of the bearing slide 170 relative to the plate 156, the two knurled nuts 174 are unscrewed and the bearing slide 170 displaced relative to the plate 156 until a desired position is reached. The bearing slide 170 can then be clamped on the plate 156 again by means of the knurled nuts 174.

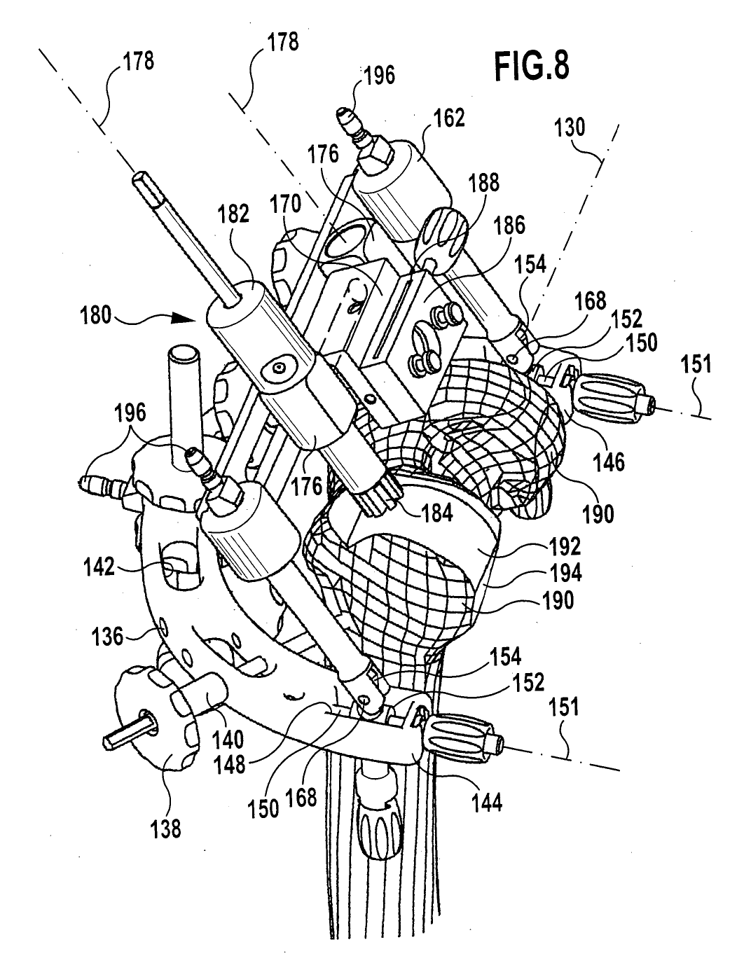

Two guide sleeves 176 are arranged laterally on the bearing slide 170 with each facing in the direction towards one of the two cylinders 162. Longitudinal axes 178 of the guide sleeves 176 extend at right angles to the pivot axis 130. A machining tool, for example, in the form of the milling cutter 180 shown in FIGS. 7 and 8, may be inserted into each of the two guide sleeves 176.

To avoid rubbing of the milling cutter 180 on the guide sleeve 176, the milling cutter 180 is, in turn, arranged on a rotary bearing 182, preferably by means of ball bearings, and the rotary bearing 182 is supported on a front face of the guide sleeve 176. The milling cutter 180 has a cylindrical work area 184 at its end, so that it may be used as face milling cutter and as side milling cutter.

A template 186 facing away from the plate 156 is arranged on the bearing slide 170 and has a guide slot 188 extending through the template parallel to the longitudinal axes 178. It serves, for example, as saw template for guiding a saw blade which is not shown.

With the aligning instrument 120, surfaces concentric with the pivot axis 130 may be prepared on the femur 124 or on any other bone of the human body. For this purpose, the platform 122 is fixed on the bone pins 126 and 128 on the femur 124, as shown in FIGS. 7 and 8. The bone pins 126 and 128 are anchored at locations on the femur 124 at which neither tendons nor muscles have grown. With the aligning instrument 120, a cylindrical anchoring surface 192 may be prepared on one of the two condyles 190 by the milling cutter 180 being inserted into one of the two guide sleeves 176 and axially immovably fixed there. When the pivot bracket 132 is pivoted about the pivot axis 130, the condyle 190 is partially resected during rotation of the work area 184 of the milling cutter 180 operating as face milling cutter. There then remains the cylindrical anchoring surface 192 on which an artificial condyle, not shown, may be anchored. If necessary, a flat cut may also be made on the condyle 190 with the aid of the template 186, whereby a flat cut surface 194 is produced on the condyle 190. The cut surface 194 extends parallel to the pivot axis 130.

For navigationally assisted use of the aligning instruments 10 and 120, these may be provided with coupling pins 196 for fixing detectable marker elements. Three coupling pins 196 protruding from the cylinders 162 and the frame 134 are arranged on the aligning instrument.

1. Surgical positioning and holding device for positioning and holding a guide for a surgical machining tool, comprising at least one fastening element for fixation to a bone to be machined and a platform held on the at least one fastening element so as to hold the guide, wherein the guide is mounted for rotation about a first axis of rotation and is designed such that at least one surface concentric with the first axis of rotation may be prepared with the machining tool guided in or held on the guide.

2. Device in accordance with claim 1, wherein a second fastening element is provided for fixation to the bone to be machined, and the second fastening element is guided and/or held on the platform.

3. Device in accordance with claim 1, wherein the platform comprises at least one fastening element receptacle for receiving the at least one fastening element.

4. Device in accordance with claim 3, wherein the at least one fastening element receptacle comprises a bore.

5. Device in accordance with claim 3, wherein longitudinal axes of at least two fastening element receptacles are aligned parallel to each other.

6. Device in accordance with claim 5, wherein the longitudinal axes of the at least two fastening element receptacles extend parallel or almost parallel to the first axis of rotation.

7. Device in accordance with claim 1, wherein a bearing shaft is provided on the platform, and the bearing shaft defines the first axis of rotation.

8. Device in accordance with claim 7, wherein the bearing shaft is mounted on the platform so as to be displaceable in a first direction of displacement relative to the at least one fastening element.

9. Device in accordance with claim 7, wherein the bearing shaft is mounted on the platform so as to be displaceable in a second direction of displacement relative to the at least one fastening element.

10. Device in accordance with claim 7, wherein the bearing shaft is mounted on the platform so as to be pivotable about a second axis of rotation relative to the at least one fastening element.

11. Device in accordance with claim 10, wherein the first and second axes of rotation are oriented at right angles to each other.

12. Device in accordance with claim 7, wherein the device comprises an articulated arm mounted for rotation about the first axis of rotation, one end of the articulated arm is mounted on the bearing shaft so as to be rotatable about the first axis of rotation, and another end of the articulated arm carries the guide.

13. Device in accordance with claim 1, wherein the guide comprises a sleeve for receiving the machining tool.

14. Device in accordance with claim 13, wherein the sleeve is mounted for rotation on the articulated arm.

15. Device in accordance with claim 7, wherein a first linear drive is provided on the platform in order to displace the bearing shaft in the first direction of displacement relative to the at least one fastening element.

16. Device in accordance with claim 15, wherein the first linear drive is a spindle drive with a first threaded spindle and a first drive knob, and a longitudinal axis of the first threaded spindle defines the first direction of displacement.

17. Device in accordance with claim 9, wherein a second linear drive is provided on the platform in order to displace the bearing shaft in the second direction of displacement relative to the at least one fastening element.

18. Device in accordance with claim 17, wherein the second linear drive is a second spindle drive with a second threaded spindle and a second drive knob, and a longitudinal axis of the second threaded spindle defines the second direction of displacement.

19. Device in accordance with claim 18, wherein the longitudinal axis of the second threaded spindle defines the second axis of rotation.

20. Device in accordance with claim 10, wherein an eccentric drive is provided on the platform in order to pivot the bearing shaft about the second axis of rotation relative to the at least one fastening element.

21. Device in accordance with claim 20, wherein the eccentric drive comprises a rotational member mounted eccentrically about a third axis of rotation, and the third axis of rotation extends parallel to the second axis of rotation.

22. Device in accordance with claim 1, wherein the at least one fastening element is spaced from the first axis of rotation.

23. Device in accordance with claim 1, wherein the guide is securable in a rotational position relative to the platform.

24. Device in accordance with claim 1, wherein the guide defines a fourth axis of rotation.

25. Device in accordance with claim 9, wherein a width of the platform in the second direction of displacement is 30 mm at maximum.

26. Device in accordance with claim 1, wherein a spacing of the guide from the first axis of rotation lies in a range of from 15 mm to 50 mm.

27. Device in accordance with claim 12, wherein a set of articulated arms of different lengths is provided, and each articulated arm has a different spacing between the first axis of rotation and the guide.

28. Device in accordance with claim 1, wherein a reference element for navigation control is provided on the device.

29. Device in accordance with claim 1, wherein the guide is displaceable in a direction parallel to the first axis of rotation relative to the platform.

30. Device in accordance with claim 24, wherein a fourth axis of rotation defined by the guide extends at right angles to the first axis of rotation.
